(19) United States
(12) Patent Application Publication
(10) Pub. No.: US 2020/0152291 A1
(43) Pub. Date: May 14, 2020

(54) METHODS AND SYSTEMS FOR AUTOMATED SEQUENCE DETERMINATION USING PATTERN-DIRECTED ALIGNED PATTERN CLUSTERING

(71) Applicants: Andrew K. C. WONG, Waterloo (CA); Ho Yin SZE-TO, Waterloo (CA)

(72) Inventors: Andrew K. C. WONG, Waterloo (CA); Ho Yin SZE-TO, Waterloo (CA)

(21) Appl. No.: 16/679,530

(22) Filed: Nov. 11, 2019

Related U.S. Application Data
(60) Provisional application No. 62/758,773, filed on Nov. 12, 2018.

(51) Int. Cl. G16B 30/00; G06F 16/22; G16B 20/20

(52) U.S. Cl. CPC G16B 30/00; G16B 20/20; G06F 16/2282

(57) ABSTRACT

There is provided a system and method for automated sequence determination using pattern-directed aligned pattern clustering. The method includes: determining a set of seed patterns; generating an address table mapping the determined sets of seed patterns to occurrences of sequences of seed patterns in the protein nucleotide sequence; determining a breakpoint gap between two respective seed patterns in an occurrence of one of the sequences in the address table; for each sequence of seed patterns in the address table, where there is a breakpoint gap, merging the respective seed patterns in the sequence surrounding the breakpoint gap with the breakpoint gap into an extended seed pattern, otherwise merging the respective seed patterns in the sequence into an extended seed pattern; and determining rare mutant patterns from the extended seed patterns by comparing extended seed patterns.

### TECHNICAL FIELD

The following relates generally to bioinformatics; and, more particularly, to methods and systems for automated sequence determination using pattern-directed aligned pattern clustering.

### BACKGROUND

The identification of functional regions from protein nucleotide sequences is a large challenge in bioinformatics and is of fundamental importance for protein sequences analysis. The general rationale is that in the evolutionary process, functional regions normally remain conserved (intact), allowing them to be identified as base/amino acid patterns from a set of biosequences respectively. However, mutations, such as substitution, insertion, and deletion, can also occur in these functional regions. Knowledge of these mutations, if spotted effectively, have the possibility of revealing functionality and mutation hotspots. In turn, this can enable researchers and clinicians to gain a better understanding of biological mechanisms and help in the design of new drugs and curing of genetic diseases.

### SUMMARY

In an aspect, there is provided a computer-implemented method for automated sequence determination using pattern-directed aligned pattern clustering, comprising: receiving as input one or more character sequences; determining a set of seed patterns having a predetermined width from each of the character sequences; generating an address table mapping the determined sets of seed patterns to occurrences of sequences of seed patterns in each of the character sequences; determining a breakpoint gap between two respective seed patterns in an occurrence of one of the seed pattern sequences in the address table, where a breakpoint gap is present if the gap between the two seed patterns is greater than or equal to a defined non-negative integer; for each sequence of seed patterns in the address table, where there is a breakpoint gap, merging the respective seed patterns in the sequence surrounding the breakpoint gap with the breakpoint gap into an extended seed pattern, otherwise merging the respective seed patterns in the sequence into an extended seed pattern; and outputting each of the extended seed patterns.

In a particular case, the method further comprising: determining mutant patterns from the extended seed patterns by comparing extended seed patterns having at least one breakpoint gap to the extended seed patterns without at least one breakpoint gap; and outputting the mutant patterns.

In another case, the predetermined width is between two and four.

In yet another case, the predetermined width is two.

In yet another case, determining the set of seed patterns having the predetermined width comprises using a pattern discovery approach based on a suffix tree.

In yet another case, the address table comprises sequences of seed patterns only where the occurrences of those seed patterns are greater than or equal to a predetermined support threshold.

In yet another case, the predetermined support threshold is determined by determining support of the seed patterns having the predetermined width, sorting such seed patterns in descending order, and setting the predetermined support threshold to be the support of the ninetieth-percentile of the sorted seed patterns.

In yet another case, the defined non-negative integer is between zero and three.

In yet another case, mutant patterns are patterns with occurrences less than the predetermined support threshold.

In yet another case, the address table further comprises a sequence ID and a position of the seed patterns.

In yet another case, the method further comprising outputting the address table.

In yet another case, the method further comprising outputting a type of each of the mutant patterns by: where one or more of the characters in the extended seed pattern having at least one breakpoint gap are a different letter compared to the extended seed patterns without at least one breakpoint gap, outputting a substitution mutation; where one or more of the characters in the extended seed pattern having at least one breakpoint gap are missing compared to the extended seed patterns without at least one breakpoint gap, outputting a deletion mutation; and where one or more of the characters in the extended seed pattern having at least one breakpoint gap are added compared to the extended seed patterns without at least one breakpoint gap, outputting an insertion mutation.

In yet another case, the method further comprising ranking the extended seed patterns according to statistical significance.

In yet another case, the method further comprising outputting a set of growing Aligned Pattern Clusters (gAPCs) by: determining a seed gAPC as the extended seed pattern having the highest-ranking, the seed gAPC comprising the seed patterns and the mutant patterns from the extended seed pattern; inducing a data space of the seed gAPC using the seed patterns and the mutant patterns; repeatedly growing the seed patterns and the mutant patterns in the seed gAPC until a termination condition has been reached, by: if a next highest-ranking extended seed pattern is significantly similar to one or more respective gAPCs in the set of gAPCs and the occurrence of such extended seed pattern is greater than or equal to the predetermined support threshold, the extended seed pattern is included in the respective gAPC that is most similar; otherwise if the next highest-ranking extended seed pattern is significantly similar to a respective one of the gAPCs in the set of gAPCs and the occurrence of such extended seed pattern is less than the predetermined support threshold, the extended seed pattern is included in the respective gAPC that is most similar to; and otherwise the next highest-ranking extended seed pattern is included in a new seed gAPC in the set of gAPCs where the new seed gAPC comprises the extended seed patterns and the mutant patterns from the next highest-ranking extended seed pattern; and outputting the set of gAPCs.

In yet another case, significant similarity is determined as having a p-value less than or equal to 0.05.

In another aspect, there is provided a system for automated sequence determination using pattern-directed aligned pattern clustering, the system comprising one or more processors and one or more storage devices storing instructions that, when executed by the one or more processors, cause the one or more processors to execute: an input module to receive as input one or more character sequences; a pattern module to determine a set of seed patterns having a predetermined width, to generate an address table mapping the determined sets of seed patterns to occurrences of sequences of seed patterns in each of the character sequences where the occurrences are greater than or equal to a predetermined support threshold, and to determine a breakpoint gap between two respective seed patterns in an occurrence of one of the sequences in the address table, where a breakpoint gap is present if the gap between the two seed patterns is greater than or equal to a defined non-negative integer; an extension module to, for each sequence of seed patterns in the address table, where there is a breakpoint gap, merge the respective seed patterns in the sequence surrounding the breakpoint gap with the breakpoint gap into an extended seed pattern, otherwise merging the respective seed patterns in the sequence into an extended seed pattern; and an output module to output each of the extended seed patterns.

In a particular case, the extension module further determines mutant patterns from the extended seed patterns by comparing extended seed patterns having at least one breakpoint gap to the extended seed patterns without at least one breakpoint gap, and the output module further outputs the mutant patterns.

In another case, the predetermined width is between two and four.

In yet another case, the predetermined width is two.

In yet another case, determining the set of seed patterns having the predetermined width comprises using a pattern discovery approach based on a suffix tree.

In yet another case, the address table comprises sequences of seed patterns only where the occurrences of those seed patterns are greater than or equal to a predetermined support threshold.

In yet another case, the predetermined support threshold is determined by determining support of the seed patterns having the predetermined width, sorting such seed patterns in descending order, and setting the predetermined support threshold to be the support of the ninetieth-percentile of the sorted seed patterns.

In yet another case, the defined non-negative integer is between zero and three.

In yet another case, mutant patterns are patterns with occurrences less than the predetermined support threshold.

In yet another case, the address table further comprises a sequence ID and a position of the seed patterns.

In yet another case, the output module further outputs the address table.

In yet another case, the extension module further outputs a type of each of the mutant patterns by: where one or more of the characters in the extended seed pattern having at least one breakpoint gap are a different letter compared to the extended seed patterns without at least one breakpoint gap, outputting a substitution mutation; where one or more of the characters in the extended seed pattern having at least one breakpoint gap are missing compared to the extended seed patterns without at least one breakpoint gap, outputting a deletion mutation; and where one or more of the characters in the extended seed pattern having at least one breakpoint gap are added compared to the extended seed patterns without at least one breakpoint gap, outputting an insertion mutation.

In yet another case, the extension module further ranks the extended seed patterns according to statistical significance.

In yet another case, the one or more processors further execute a gAPC module to output a set of growing Aligned Pattern Cluster (gAPCs) by: determining a seed gAPC as the extended seed pattern having the highest-ranking, the seed gAPC comprising the seed patterns and the mutant patterns from the extended seed pattern; inducing a data space of the seed gAPC using the seed patterns and the mutant patterns; and repeatedly growing the seed patterns and the mutant patterns in the seed gAPC until a termination condition has been reached, by: if a next highest-ranking extended seed pattern is significantly similar to one or more respective gAPCs in the set of gAPCs and the occurrence of such extended seed pattern is greater than or equal to the predetermined support threshold, the extended seed pattern is included in the respective gAPC that is most similar; otherwise if the next highest-ranking extended seed pattern is significantly similar to a respective one of the gAPCs in the set of gAPCs and the occurrence of such extended seed pattern is less than the predetermined support threshold, the extended seed pattern is included in the respective gAPC that is most similar to; and otherwise the next highest-ranking extended seed pattern is included in a new seed gAPC in the set of gAPCs where the new seed gAPC comprises the extended seed patterns and the mutant patterns from the next highest-ranking extended seed pattern, wherein the output module further outputs the gAPC.

In yet another case, significant similarity is determined as having a p-value less than or equal to 0.05.

These and other aspects are contemplated and described herein. It will be appreciated that the foregoing summary sets out representative aspects of methods and systems for producing an expanded training set for machine learning using biological sequences to assist skilled readers in understanding the following detailed description.

### DESCRIPTION OF THE DRAWINGS

The features of the invention will become more apparent in the following detailed description in which reference is made to the appended drawings wherein:

FIG. 1 is a diagram illustrating a system for automated sequence determination using pattern-directed aligned pattern clustering in accordance with an embodiment;

FIG. 2 is a flow chart illustrating a method for automated sequence determination using pattern-directed aligned pattern clustering;

FIG. 3A is an example input for the system of FIG. 1;

FIG. 3B is the example input of FIG. 3A showing functional patterns;

FIG. 3C is an example output for the system of FIG. 1;

FIG. 4 is an example diagrammatic overview of the workflow of the system of FIG. 1;

FIG. 5A is an example diagrammatic workflow of the system of FIG. 1 showing a substitution mutation;

FIG. 5B is an example diagrammatic workflow of the system of FIG. 1 showing an insertion mutation;

FIG. 5C is an example diagrammatic workflow of the system of FIG. 1 showing an deletion mutation;

FIG. 6A is an example diagrammatic workflow of the system of FIG. 1 showing a determination of model width for a seed width of 3;

FIG. 6B is an example diagrammatic workflow of the system of FIG. 1 showing a determination of model width for a seed width of 4;

FIG. 7 is an illustration of an example comparison of an MEME approach, an APCn approach, and the system of FIG. 1;

FIG. 8 is a graphical illustration of a definition of true-positive, false-positive and false-negative for an example quantitative evaluation of predicted conserved regions;

FIG. 9A is a chart showing a first APC obtained from an example experiment of the system of FIG. 1 on a Cytochrome C dataset;

FIG. 9B is a chart showing a second APC obtained from the example experiment of the system of FIG. 1 on the Cytochrome C dataset;

FIG. 9C is a chart showing a third APC obtained from the example experiment of the system of FIG. 1 on the Cytochrome C dataset;

FIG. 10A is a chart showing a first APC obtained from an example experiment of the system of FIG. 1 on an Ubiquitin dataset;

FIG. 10B is a chart showing a second APC obtained from an example experiment of the system of FIG. 1 on the Ubiquitin dataset;

FIG. 10C is a chart showing a third APC obtained from an example experiment of the system of FIG. 1 on the Ubiquitin dataset; and

FIG. 10D is a chart showing a fourth APC obtained from an example experiment of the system of FIG. 1 on the Ubiquitin dataset.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

A protein sequence usually consists of a number of functional regions, generally varying in width from 25 to 500 amino acids. Under evolutionary pressure, these regions normally remain conserved. To identify them, one approach called domain annotation leverages existing databases (such as PFam) or profile hidden markov models.

For de novo discovery of functional regions, Multiple Sequence Alignment (MSA) is one approach that can be used, but it is generally suitable only for globally homologous sequences with a high level of similarity. Even within the same protein family, this “homologous” assumption may not hold. For example, in the class A Scavenger Receptor with five subclasses, the width of collagenous domains varies in subclasses from 75 to 250 amino acids.

Motif discovery is another approach that can be used to locate and align locally homologous sub-sequences to obtain a position-weight matrix (PWM), which is a fixed-length representation model; but where the span of protein functional regions, with frameshifts (insertion and deletion mutations) varies in width. PWM thus requires computational expensive exhaustive searches to obtain a PWM with width of optimal range. For example, in Multiple Em for Motif Elicitation (MEME), the search range of the default PWM width parameter generally varies from 8 to 50. Thus, approaches to identifying functional regions of protein sequences, such as those based on PWMs, generally have to assume or confine functional regions having a fixed width due to computational concerns. Furthermore, with such constraint, such approaches generally cannot identify functional regions with minor mutations, particularly those with insertion or deletion mutations. Additionally, it may take exhaustive search to find an optimal width.

A particular approach, Aligned Pattern Clustering (APCn), can be used to identify functional regions by grouping and aligning patterns with variable width from protein family sequences as Aligned Pattern Clusters (APCs). APC can be useful due to its dual space representation, consisting of the pattern and the data space. The former displays the aligned patterns with statistical significance measures and supports (the “what” and their statistical significance); the latter displays all the patterns in the APC on the original sequence space, the “where” and the delimited range of the domain covering all its patterns. Nevertheless, if certain mutations such as substitution, insertion and/or deletion occur in a small subset of sequences, APCn generally cannot include them in the discovered functional regions because their frequencies of occurrence are too low to be considered as patterns.

The present embodiments, advantageously, are intended to overcome challenges of other approaches using what the present inventors refer to as Pattern-Directed Aligned Pattern Clustering (PD-APCn). The presently described embodiments are generally also applicable to domains in which a real-valued data stream can be discretized into a character stream of data in order to identify patterns in that data, even if such patterns are interrupted by varying intermediary characters or variable length strings of intermediary characters. Examples of such domains include, but are not limited to, analysis of protein nucleotide sequences, cybersecurity, insurance, finance, etc.

By discovering seed patterns from input sequence data, with sequence positions located and recorded on an address table, PD-APCn can use the seed patterns to direct incremental extension of functional regions; for example, those with minor mutations. By grouping aligned extended patterns, PD-APCn can recruit patterns adaptively and efficiently with variable width without relying on exhaustive optimal search under width parameter tuning. The present inventors conducted example experiments on synthetic datasets, with different sizes and noise levels, and showed that PD-APCn can identify implanted patterns with mutations. Advantageously, PD-APCn was shown to outperform other approaches, for example, the motif-finding software, Multiple Em for Motif Elicitation (MEME); which uses PWMs. PD-APCn was shown to have much higher recall and Fmeasure, and an approximate measured computational increase of 665 times faster than MEME. As an example, when applying PD-APCn on datasets from Cytochrome C and Ubiquitin protein families, all key binding sites conserved in the families (as reported in the literature) were captured in the Aligned Pattern Clustering (APC) outputs.

In this way, PD-APCn can incrementally recruit new statistically significant patterns into an APC during the APC expansion process while placing the mutation patterns, which as a whole may not be statistical significant, into a pool of mutants. In embodiments, the pool of identified mutants could be considered as “rare mutants” given their lack of statistical significant occurrence. Nevertheless, the term “rare” as used herein is not to be construed as being limited to any particular statistical threshold and is merely used as a convenient descriptor. Concurrently or sequentially, PD-APCn can also track their positions in the data space for future exploration and referral. Advantageously, PD-APCn can perform these tasks in a unified approach.

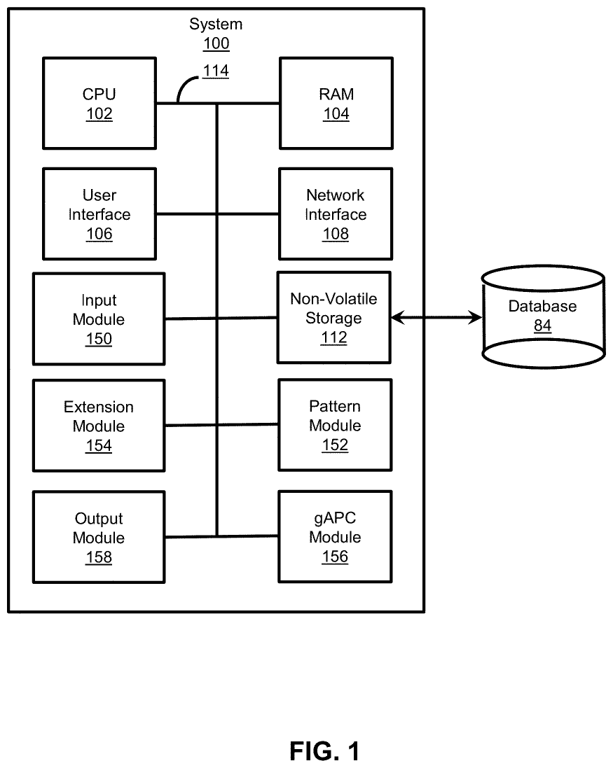

Referring now to FIG. 1, a system 100 for sequence determination using pattern-directed aligned pattern clustering, in accordance with an embodiment, is shown. In this embodiment, the system 100 is run on a local computing device. In further embodiments, the local computing device can have access to content located on a server over a network, such as the Internet. In further embodiments, the system 100 can be run on any suitable computing device; for example, the server. In some embodiments, the components of the system 100 are stored by and executed on a single computing device. In other embodiments, the components of the system 100 are distributed among two or more computing devices that may be locally or remotely distributed.

FIG. 1 shows various physical and logical components of an embodiment of the system 100. As shown, the system 100 has a number of physical and logical components, including a central processing unit (“CPU”) 102 (comprising one or more processors), random access memory (“RAM”) 104, a user interface 106, a network interface 108, non-volatile storage 112, and a local bus 114 enabling CPU 102 to communicate with the other components. In some cases, at least some of the one or more processors can be graphical processing units. CPU 102 executes an operating system, and various modules, as described below in greater detail. RAM 104 provides relatively responsive volatile storage to CPU 102. The user interface 106 enables an administrator or user to provide input via an input device, for example a keyboard and mouse. The user interface 106 can also output information to output devices to the user, such as a display and/or speakers. The network interface 108 permits communication with other systems, such as other computing devices and servers remotely located from the system 100, such as for a typical cloud-based access model. Non-volatile storage 112 stores the operating system and programs, including computer-executable instructions for implementing the operating system and modules, as well as any data used by these services. Additional stored data can be stored in a database 84. During operation of the system 100, the operating system, the modules, and the related data may be retrieved from the non-volatile storage 112 and placed in RAM 104 to facilitate execution.

In an embodiment, the system 100 further includes one or more conceptual modules executed on the CPU 102. In this embodiment, the system 100 includes an input module 150, a pattern module 152, an extension module 154, an GAPC module 156, and an output module 158.

In some cases, the functions and/or operations of the modules can be combined or executed on other modules.

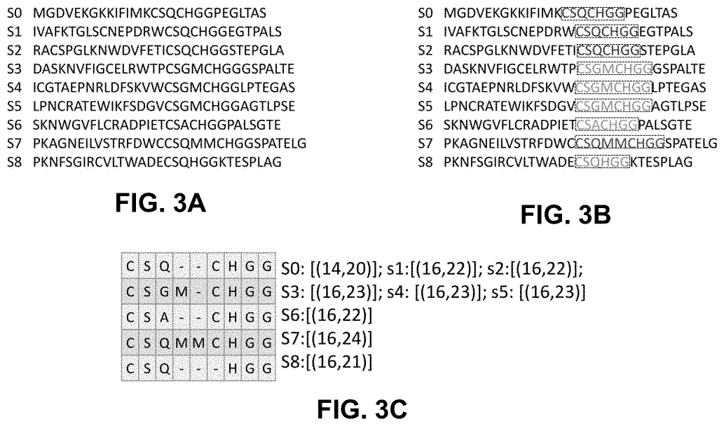

A simplified example of inputs and outputs of an example PD-APCn for the system 100 are shown in FIGS. 3A to 3C. As shown in FIG. 3A, the input can include a set of sequences within the same family exhibiting homologous biological functions. FIG. 3B illustrates the implanted patterns in the data set. In this case, as illustrated in FIG. 3C, the outputs are: (1) starting and ending address locations of functional regions on the sequences if they exist and are discovered; and (2) a homologous site alignment of the functional regions. In this case, alignment refers to inserting gaps into a set of sequences such that vertical similarity (or site homology) is maximized. As shown in FIG. 3A, the input data can be a set of sequences (for example, S0 to S8); which is a simplified dataset having only has nine sequences (S0 to S8). In FIG. 3B, segments containing the functional patterns within a family functional region in the input data are outlined. In FIG. 3C, output data includes aligned patterns in the functional regions of a set of sequences, with their sequence IDs and starting and ending address locations determined.

With respect to FIGS. 3A to 3C, identification and alignment of functional regions with mutations from a set of sequences are illustrated. Given, as input, a set of sequences within the same family, and/or demonstrating similar biological functions, outputs of PD-APCn can be segments in the functional region containing patterns with homologous sites aligned, in some cases in addition to, starting and ending address locations of aligned patterns (if they exist in the functional region on the sequences). In a particular case, the output data can include mutated patterns with sites aligned in aligned functional regions of a set of sequences, together with their sequence IDs, starting and ending address locations labelled and displayed.

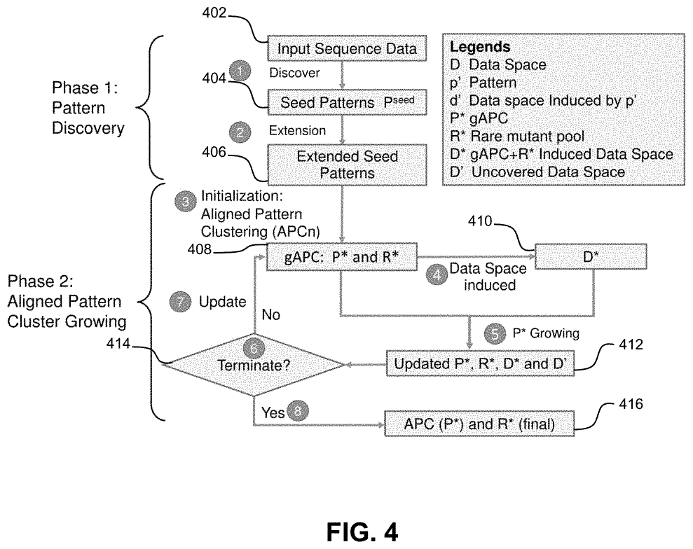

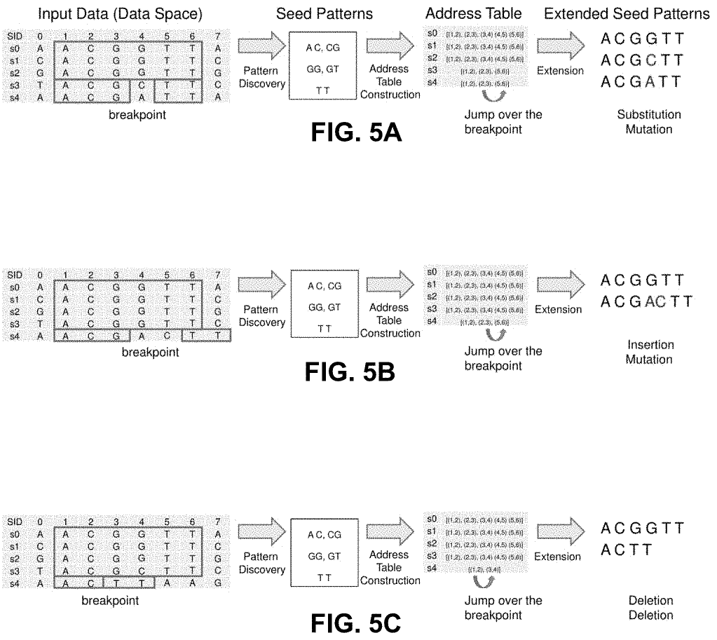

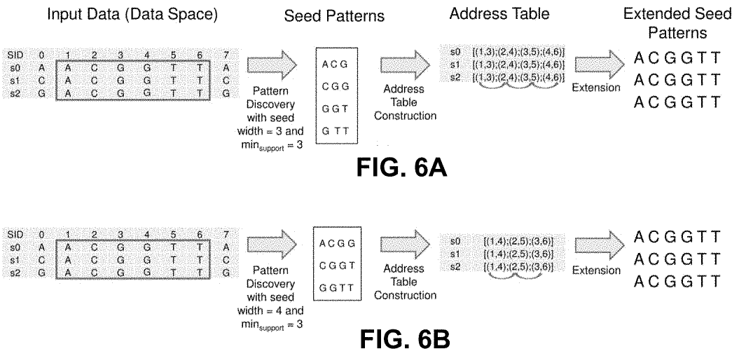

In an embodiment, there can be two phases in PD-APCn. Given a set of sequences, a first phase (“Phase 1” or “Phase I”) can be for discovery of seed patterns using a pattern discovery approach based on a suffix tree. An address table can be constructed from the seed patterns after the pattern discovery process. The seed patterns can be extended via the address table to obtain a set of extended seed patterns. Given a set of seed patterns, a second phase (“Phase 2” or “Phase II”) of PD-APCn can initiate and expand the APCs via an approach called APC growing. FIG. 4 provides an example diagrammatic overview of PD-APCn. FIGS. 5A TO 5C diagrammatically illustrate pattern breakpoint discovery and its use for discovering patterns with mutations. FIGS. 6A and 6B diagrammatically illustrate extension of seed patterns to adaptively determine a representation model width.

For the purposes of the following disclosure, APC stands for Aligned Pattern Cluster. APCs are a cluster (set) of patterns with alignment. Alignment is an approach to inserting gaps into a set of patterns to maximize column-wise similarity. APC can be useful because, for example, it (1) has variable width, (2) allows variants, and (3) is knowledge-rich as it has no information loss.

Turning to FIG. 4, an example diagrammatic overview of PD-APCn, according to embodiments described herein, is shown with an example workflow given in circled steps. During Phase I of pattern discovery, at block 402, input sequence data is received by the input module 150. At block 404, the pattern module 152 determines a set of seed patterns with a given pattern width (preferably small) via the pattern discovery approach (PDA) based on a suffix tree. At block 406, the extension module 154 extends the seed patterns to their superpatterns over breakpoint gaps, discovered via pattern breakpoint discovery as described herein, to obtain a set of extended seed patterns. During Phase II of growing of “growing APCs” (gAPCs), at block 408, the GAPC module 156 determines a seed gAPC from the extended seed patterns. Specifically, the top extended seed pattern is initially considered as a gAPC with only one pattern. Within each gAPC C*, the patterns (whose support no smaller than minSupport) are denoted as P* and rare mutational patterns (whose support smaller than minSupport) are denoted as R*. At block 410, data space D is induced from P* and R* via, at block 412, the suffix tree. For a next extended seed pattern p′, the system 20 performs: if p′ is found significantly similar to the patterns in a gAPC C*, and its support is no smaller than minSupport, it is included in P*, and then P*, D* and D are updated; if p′ is found significantly similar to the patterns in a gAPC C*, and its support is smaller than minSupport, it is included in R*, and R*, D* and D are updated; and otherwise, p′ is considered as a new gAPC with only one pattern. At block 414, a terminating condition is checked; for example, if a specified number of extended seed patterns is reached. If the terminating condition is false, APC growing is performed again at block 408. If the terminating condition is true, at block 416, the gAPC is taken as a final model, which is composed of APC (P*) and R*. In this way, the final models can be ranked based on their support. In some cases, the final models with highest rankings can be outputted by the output module 158.

Turning to FIGS. 5A to 5C, an example diagram showing use of pattern breakpoints for discovering three types of mutation patterns is shown. In this case, the three types of mutations are: substitution shown in FIG. 5A, insertion shown in FIG. 5B, and deletion shown in FIG. 5C. The identification of each seed pattern can be configured to be dependent on a configured seed width, being the number of adjacent (consecutive) bases (i.e., A, C, G or T) in the seed pattern, and the minimum number of times each such pattern appears across the set of input sequences to be considered a seed pattern (referred to herein as “minSupport”).

In this example, with the seed width=2 and minSupport=5) the pattern module 152 discovers seed patterns from the sequences comprising the input data (data space). An address table is constructed from the occurrence of the discovered seed patterns. For each seed pattern, one or more sub-pattern breakpoints are discovered using the address table. The pattern module 152 determines the breakpoints by locating for each sequence the locations that do not consist of a seed pattern, which is exposed in the address table when adjacent sequence positions are not represented by one of the seed patterns (such as {3,4} and {4,5} in both s3 and s4 of FIG. 5A). By jumping over the breakpoints between the sub-patterns, a set of extended seed patterns with breakpoint gap (gapbreak=2), encompassing the rare mutational patterns, can be discovered via seed pattern extension.

Some mutated patterns (when fragmented) may not be discovered by the pattern discovery approach (PDA) since the frequency of occurrences of the entire mutational pattern is too low. In FIG. 5A, in the input data space, a pattern ACGGTT occurs 3 times over 5 sequences. However, its mutated variants ACGCTT and ACGATT, with a single substitution mutation, occur only once and thus cannot be discovered statistically as patterns. Nevertheless, the sub-patterns ACG and TT may still have high frequency of occurrences (if functional), and thus they can still be discovered as patterns using the present embodiments. Hence, using the address location of the sub-patterns ACG and TT, the mutation spot between them (in this example, C and A) can be considered as a breakpoint. By jumping over it, the mutated variants ACGCTT and ACGATT can be discovered. In a similar manner, FIG. 5B and 5C illustrate the finding of the insertion and deletion mutations, respectively, through the use of breakpoints.

Turning to FIGS. 6A to 6B, an example diagram showing extension of seed patterns to adaptively determine a representation model width. Seed patterns are first discovered from the input data (data space). In the example of FIG. 6A, with a seed width=3, and minSupport=3. In the example of FIG. 6B, with a seed width=4, and minSupport=3. An address table is constructed from the occurrence of the discovered patterns. By jumping over the breakpoints between the pattern occurrence, a set of extended seed patterns can be discovered. Advantageously, it can be observed that the set of extended seed patterns obtained in FIGS. 6A and 6B respectively are the same, illustrating that the representation model width can be obtained from data adaptively without having to resort to exhaustive search.

The present embodiments of PD-APCn can use seed pattern extension to increase the coverage of the growing APC. The width of seed patterns is generally inherent in the input data and should not be affected by the process and/or the width parameters. As shown in FIG. 6A, with a seed width=3, the approach of jumping over a breakpoint and obtaining a full coverage is applied. When the seed width is changed to 4 (FIG. 6B), the same full coverage is obtained, showing pattern width adaptation without exhaustive search.

Leveraging pattern discovery approach (PDA) based on a suffix tree, the system 100 can advantageously discover patterns with any width specified, locate the pattern occurrence, and count the pattern support. Hence, the system 100 can obtain a set of patterns to serve as seeds efficiently. Such information can be used, for example, to find breakpoints where mutated patterns can be identified.

In the present disclosure, a suffix tree T can be considered as a function that retrieves an occurrence position of a sequence p. In an illustrative example, given a set of input sequences S as follows:

| Sequence ID | Sequence (position starts from 0) |
|---|---|
| s0 | aaaHELLObbbHELLOccc |
| s1 | ddHELLOeeee |
| s2 | fHELLOgggggggggggggg |
| s3 | hhhhhhhHELLLOkkkkkk |

If p=‘HELLO’, using the suffix tree, the occurrence of p can be determined as

T(p)=s0: [(3,7)1(11,15)]; s1: [(2,6)]; s2: [(1,5)].

By counting on T(p), it can be determined that there are 4 occurrences of P:

Occurrence(P,S)=Occurrence(T(P))=4.

Support is a more restricted measure of occurrence, as support considers the multiple occurrence of a pattern on the same sequence as only 1 count. Therefore, in the above example, although there are 2 occurrences of “HELLO” on s0, its support count is only 1:

Support(T(P))=3.

In an example, discovery of seed patterns using a pattern discovery approach based on a suffix tree can include: (1) constructing a generalized suffix tree T from a set of input sequences S; (2) segmenting the input sequences into subsequences having a particular width equivalent to a predetermined minimum width (min_{width}) (for example, if min_{width}=2, segment “APPLE” into [“AP”, “PP”, “PL”, “LE”]); (3) using the generalized suffix tree T, determining support for each subsequence; and (4) extracting the subsequences having a support value that is greater than or equal to a predetermined minimum value of support (support≥minₛᵤₚₚₒᵣₜ)

In an example implementation of the present embodiments, let Σ be a set of alphabets. Let sₖ be a sequence comprising of alphabets in Σ, i.e. sₖ=sₖ¹sₖ² . . . sₖ^{|s}^{ₖ}^{|}, where sₖ^{j}∈Σ, ∀j=1,2, . . . , |sₖ|. Let S be a set of sequences, i.e. S={sₖ|k=1,2, . . . , |S|}.

A sequences s̅ occurs in a sequence s if and only if s̅ is a subsequence of s, i.e. [symbol] i such that s̅=s[i, i+|s̅|−1], where 1≤i≤|s|−|s̅|+1. It can also be equivalent to saying that s̅ occurs at the position i in s. Hence, given a sequence segments s̅ and a sequence s, the occurrence of s̅ in s is defined as:

$$Occurrence ( \overline{s} , s ) = ( \begin{matrix}1 , & if \overline{s} occurs in s \\ 0 , & otherwise \end{matrix} \qquad ( 1 )$$

Given a sequence s̅, and a set of sequences S, the support of s̅ over S is defined as the number of sequences in S in which s̅ occurs. Formally:

Support(s̅, S)=Σₛ_{ₖ}_{∈s}Occurrence(s̅, sₖ) (2)

Given a set of sequences S, a sequence p is considered as a pattern if its support is larger than or equal to a minimum threshold minₛᵤₚₚₒᵣₜ, i.e. Support(p, S)≤minₛᵤₚₚₒᵣₜ . A seed pattern p̅ is defined as a pattern with a particular width w_{seed}, i.e. |p̅|=w_{seed}. Given a set of sequences S, a set of seed patterns p^{seed} can then be discovered from S by the pattern discovery approach via setting w_{seed} and minₛᵤₚₚₒᵣₜ, i.e. p^{seed}={p̅ⁱ|i=1, . . . , |P̅|}={p̅¹, p̅², . . . , p̅^{|P̅|}}.

Given a set of sequences S and a set of Patterns P, a sequence r is considered as a rare mutant pattern if its support is lower than a minimum threshold minₛᵤₚₚₒᵣₜ, i.e. Support(p, S)<min_{Support} and is found to be significantly similar to the patterns in P, i.e. ALIGN(P, r)≥min_{Similarity}.

Given a set of patterns P̅^{l}={p̅^{l,1},p̅^{l,2}, . . . , p̅^{l,m}^{ₗ}}, an APC C^{l} is defined as:

$$\begin{matrix}{C}^{l} = ALIGN ( {\overline{P}}^{l} ) & ( 3 ) \\ = ALIGN ( \begin{matrix}{\overline{p}}^{l , 1} \\ {\overline{p}}^{l , 2} \\ ⋮ \\ {\overline{p}}^{l , {m}_{l}}\end{matrix} ) = ( \begin{matrix}{p}^{l , 1} \\ {p}^{l , 2} \\ ⋮ \\ {p}^{l , {m}_{l}}\end{matrix} ) = ( {P}^{l} ) & ( 4 ) \\ = {( \begin{matrix}{σ}_{1}^{l , 1} & {σ}_{2}^{l , 1} & & {σ}_{{n}_{l}}^{l , 1} \\ {σ}_{1}^{l , 2} & {σ}_{2}^{l , 2} & & {σ}_{{n}_{l}}^{l , 2} \\ ⋮ & ⋮ & ⋮ & ⋮ \\ {σ}_{1}^{l , {m}_{l}} & {σ}_{2}^{l , {m}_{l}} & & {σ}_{{n}_{l}}^{l , {m}_{l}}\end{matrix} )}_{{m}_{l} \times {n}_{l}} , & ( 5 )\end{matrix}$$

where σⱼ^{l,i}∈Σ∪{−}, ∀i=1,2, . . . , mₗ, ∀j=1,2, . . . , nₗ, and ALIGN is a process to maximize the column similarity in p̅^{l}, by inserting gaps, to obtain a set of aligned patterns p^{l}={p^{l,1},p^{1,2}, . . . ,^{l,m}^{ₗ}} with the same length nₗ. Implementation of the ALIGN process would be apparent to a skilled person.

Thus, for example, given a set of sequences S={sₖ|k=1,2, . . . , |S|}, a positive integer w_{seed}∈ [symbol] ₊ to determine the width of seed patterns, a positive integer minₛᵤₚₚₒᵣₜ∈ [symbol] ₊ to as the predetermined support threshold of seed patterns, a positive integer gap_{break}∈ [symbol] ₊ to control the breakpoint gap, and a real-valued similarity threshold min_{Similarity}∈ [symbol] to cluster patterns, the system 20 endeavours to determine a set of aligned pattern clusters (APCs) [symbol] ={C^{l}|l=1, . . . , | [symbol] }={C¹,C², . . . , [symbol] , [symbol] }.

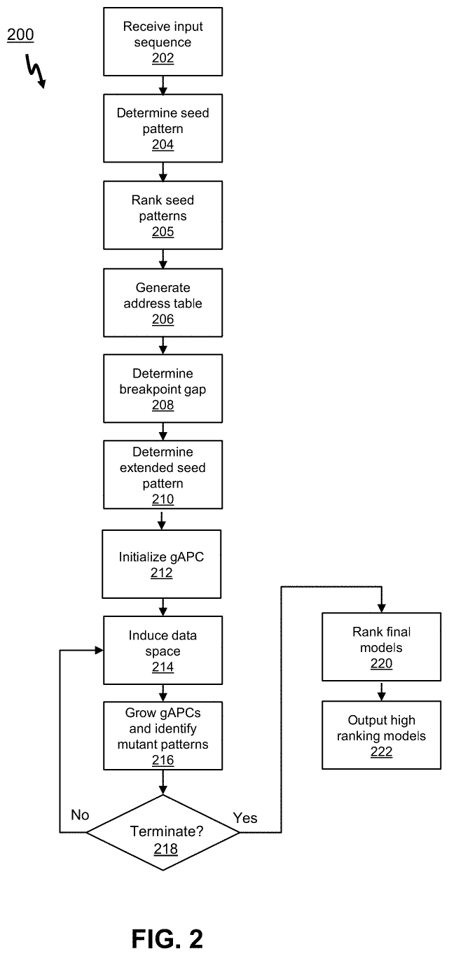

Turning to FIG. 2, a method for sequence determination using pattern-directed aligned pattern clustering 200, according to an embodiment, is shown.

At block 202, the input sequence (data space) is received by the input module 150 from the database 84 or from another computing device via the network interface 72.

At block 204, the pattern module 152 can determine patterns with a specified width, locate the pattern occurrence, and count the pattern support. In a particular case, the pattern module 152 can do so using a pattern discovery approach (PDA) based on a suffix tree, as described herein. In a particular case, the specified width can be between two and four; and advantageously in the present embodiments for sequence determination, can be as small as two. Hence, a set of patterns can be efficiently obtained to serve as seeds. At block 205, in some cases, the pattern module 152 can rank the determined seed patterns according to their support from highest to lowest. Such information can be used later to assist in finding breakpoints where mutated patterns can be identified. In some cases, during PDA, delta-close redundancy and statistical non-induce pruning can be turned off.

Using the PDA based on the suffix tree, given a seed pattern p̅^{j}, the system 100 can retrieve sequences in which p̅^{j} occurs and its occurrence positions. For example, as shown in FIG. 5A, the occurrence of ACGGTT over s1 is (1,6). Hence, an address table mapping a sequence sₖ to the occurrence of seed patterns on itself can be constructed.

At block 206, the extension module 154 generates an address table. Given a sequence sₖ, and a set of seed patterns P^{seed}, a function H is defined as follows:

H(sₖ,P^{seed})={(o₁^{k},t₁^{k}), (o₂^{k},t₂^{k}), . . . ,(oₙ_{ₖ}^{k},tₙ_{ₖ}^{k})} (6)

where oⱼ^{k} is the position that a seed pattern p̅^{j}∈P^{seed} occurs in sₖ, tⱼ^{k} is the ending position, ∀j=1,2, . . . , nₖ, and nₖ is the number of seed patterns occurring in sₖ. For example, as shown in FIG. 5A, H(s₃, {AC, CG, GG, GT, TT})={(1,2), (2,3), (3,6)}. An address table is constructed by the extension module 154 by applying function H to every sₖ∈S.

In some cases, the address table only lists the seed patterns above the predetermined support threshold (minₛᵤₚₚₒᵣₜ). In a particular case, the predetermined support threshold is determined by determining support of the seed patterns having the predetermined width, sorting such seed patterns in descending order, and setting the predetermined support threshold to be the support of the ninetieth-percentile of the sorted seed patterns

At block 208, the extension module 154 determines breakpoint gaps. Given two pattern occurrences, (oᵢ^{k}, tᵢ^{k}) and (oᵢ₊₁^{k}, tᵢ₊₁^{k}), the gap between them is defined as:

gap₍ₒ_{ᵢ}_{^{k}}_{,t}_{ᵢ}k_{),(o}_{_{i−1}}_{^{k}}_{,t}_{ᵢ₊₁}_{^{k}}₎=oᵢ₊₁^{k}−tᵢ^{k}−1 (7)

Where, in some cases, the two pattern occurrences, (oᵢ^{k}, tᵢ^{k}) and (oᵢ₊₁^{k}, tᵢ₊₁^{k}) could be merged into one pattern occurrence (oᵢ^{k}, tᵢ₊₁^{k}) , if gap₍ₒ_{ₖ}_{^{k}}_{,t}_{ᵢ}_{^{k}}_{),(o}_{ᵢ₊₁}_{^{k}}_{,t}_{ᵢ₊₁}_{^{k}}₎≤gap_{break}; where gap_{break} is a defined non-negative integer. In this way, gap₍ₒ_{ₖ}_{^{k}}_{,t}_{ᵢ}_{^{k}}_{),(o}_{ᵢ₊₁}_{^{k}}_{,t}_{ᵢ₊₁}k₎ is a breakpoint gap if gap₍ₒ_{ₖ}_{^{k}}_{,t}_{ᵢ}_{^{k}}_{),(o}_{ᵢ₊₁}_{^{k}}_{,t}_{ᵢ₊₁}_{^{k}}₎≤gap_{break}. While in the present embodiments gap_{break} is set as 2 or 3; in further cases, it can be set as any value between 0 and 3, and in further cases, any suitable value can be used.

At block 210, the extension module 154 determines extended seed patterns and stores the extended seed patterns in a list (an example is shown in FIG. 5A: [‘ACGGTT’, ‘ACGCTT’, ‘ACGATT’]). By merging pattern occurrences, the seed patterns are extended to their “superpatterns”, allowing the identification of rare mutant patterns (such as those with frameshifts). Whereby merging pattern occurrences is an operation to merge two brackets; for example, (2,7) and (10,12) will be merged as (2,12). In an example, as illustrated in FIG. 5C, “CAQHGC” has a width of 6 occurring at position 2 on s1, i.e. (2,7), and “CAG” has a width of 3 occurring at position 10 on s1, i.e. (10,12). With gap_{break}=2, these two occurrences would be grouped into one occurrence, i.e. (2,12), allowing the identification of the rare mutant pattern “CAQHGCGGCAG”. The extension module 154 applies such operation on the address table constructed to obtain a set of extended seed patterns pₑₓₜ^{seed}. In some cases, the extension module 154 can rank the extended seed patterns according to their statistical significance. Any suitable approach for determining statistical significance of a pattern can be used. In an example approach, the statistical significance of a sequence P is

$$\frac{{k}_{P} - E ( P )}{\sqrt{E ( P )}} ,$$

where kₚ is the number of times that a sequence P occur, and E(P) is the expected number of times that a sequence P occur, given a set of sequences.

After the determination of a set of extended seed patterns pₑₓₜ^{seed}, an iterative APC growing approach, directed by the extended seed patterns, can be performed. Advantageously, growing an APC allows the width of an APC to be self-determined, instead of relying on users to set it or having to rely on an exhaustive search. This permits, for example, the ability to remove one item needed for parameter tuning, and the ability to decrease computational time needed for exhaustive search.

At block 212, the GAPC module 156 initializes a set of “growing APCs” (gAPCs) by obtaining a seed gAPC from the extended seed patterns. In a particular case, the top extended seed pattern is initially considered as a gAPC with only one pattern. Within each gAPC, the patterns (with support no smaller than minₛᵤₚₚₒᵣₜ) are denoted as P* and the rare mutant patterns (with support smaller than minₛᵤₚₚₒᵣₜ) are denoted as R*. In most cases, initialization of gAPC is conducted only in the first iteration of the APC growing approach. In this case, as the extended seed patterns have been ranked according to their statistical significance, the “top” extended seed pattern is the one with the greatest statistical significance. As an example, where the statistical significance is measured in p-value score, the top extended pattern will be the one with the highest score.

At block 214, the GAPC module 156 induces a data space D* from P* and R*. Data space D* is a set of sequences containing the patterns in P* and R*, as well as data space D′ as a set of sequences not containing any patterns in P* (being the data space not yet uncovered). In a particular case, the data space D* can be efficiently induced using the suffix tree constructed using PDA.

At block 216, the GAPC module 156 grows the set of gAPCs until a termination condition has been reached. If a next highest-ranking extended seed pattern is significantly similar to one or more respective gAPCs in the set of gAPCs and the occurrence of such extended seed pattern is greater than or equal to the predetermined support threshold, the extended seed pattern is included in the respective gAPC that is most similar. Otherwise if the next highest-ranking extended seed pattern is significantly similar to a respective one of the gAPCs in the set of gAPCs and the occurrence of such extended seed pattern is less than the predetermined support threshold, the extended seed pattern is included in the respective gAPC that is most similar to. Otherwise the next highest-ranking extended seed pattern is included in a new seed gAPC in the set of gAPCs where the new seed gAPC comprises the seed patterns and the mutant patterns from the next highest-ranking extended seed pattern. In a implementation of the above, the GAPC module 156 grows the patterns P* and rare mutant patterns R* for each gAPC in the set of gAPCs, starting with just the seed gAPC. For the next extended seed pattern p′, if p′ is found significantly similar to the patterns in the seed gAPC (referred to as C*), and its support is no smaller than minₛᵤₚₚₒᵣₜ, the GAPC module 156 includes it in P* of the gAPC in the set of gAPCs to which it is most similar. The GAPC module 156 can then update P*, D* and D using this new inclusion. If p′ is found significantly similar to the patterns in a gAPC C*, and its support is smaller than minₛᵤₚₚₒᵣₜ, the GAPC module 156 includes it in R* of the gAPC in the set of gAPCs to which it is most similar. The GAPC module 156 can then update R*, D* and D based on this new inclusion. Otherwise, the GAPC module 156 considers p′ as a new seed gAPC with only one pattern. In some cases, the similarity between p′ and the patterns in a gAPC C* can be determined by the GAPC module 156 using ALIGN (P*∪R*∪p′). In a particular case, a significantly similar threshold can be a p-value of 0.05 or smaller; however, any suitable p-value for the circumstances can be used.

At block 218, the GAPC module 156 determines if a terminating condition has been reached. In a particular case, the terminating condition is if all extended seed patterns are reached. Another possible termination condition is that if any exiting gAPCs have more than a threshold of extended seed patterns (as an example, 5), then the GAPC module 156 may stop and quit the iterative process.

If the terminating condition was not reached, blocks 214 and 216 are repeated by the GAPC module 156.

If the terminating condition was reached, each gAPC C* in the set of gAPCs will be composed of P* and R* and can be considered as a final model being the final gAPCs that were outputted by the GAPC module 156. In some cases, at block 220, the GAPC module 156 can rank final models by their support. At block 222, the output module 158 can output the highest-ranking final model or those models with a ranking above a certain threshold. Advantageously, the patterns captured by P* are highly-likely to be conserved functional regions and the patterns captured by R* are highly-likely to be functional regions with mutations because conserved regions appear more than expected with statistical significance from a given input sequence dataset. Biomedical researchers can then use such determination to conduct confirmatory lab tests, research, drug discovery, and the like.

Other approaches, such as those that use PWMs, generally assume that the functional regions have a fixed width; and hence, they generally cannot identify functional regions with mutations whose occurrences do not allow them to emerge as statistically significant patterns, particularly those with insertion or deletion mutations. Other approaches to identify functional regions as APCs can do so by grouping and aligning patterns with variable width. However, these types of approaches generally cannot include substitution, insertion and/or deletion mutations in its discovered functional regions because the frequency of occurrences of such mutations are generally too low to be discoverable as patterns with such an approach.

The embodiments described herein, using PD-APCn, can advantageously identify functional regions with mutations such as substitution, insertion and deletion errors, even if the mutated patterns only occur one or two times in the input dataset. Further advantageously, the embodiments described herein can adaptively determine width of the functional regions, with minimal parameter tuning.

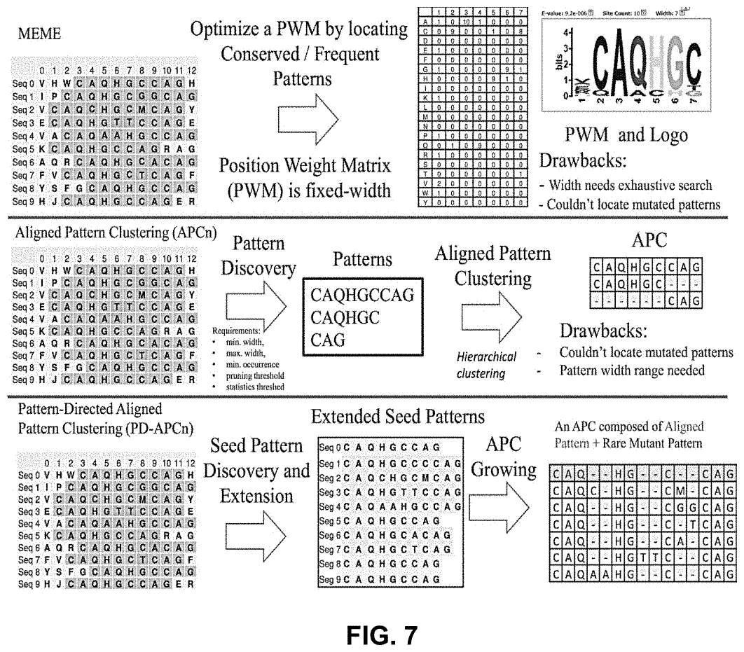

FIG. 7 diagrammatically illustrates an example comparison of an MEME approach, an APCn approach, and the PD-APCn of the present disclosure. By first discovering seed patterns from the input sequence data, with their sequence positions located and recorded on an address table, PD-APCn can use the seed patterns to direct incremental extension of functional regions including segments with minor mutations. By grouping the aligned extended patterns, PD-APCn can recruit patterns adaptively and efficiently with variable width without relying on exhaustive optimal search and parameter tuning. As shown in FIG. 7, MEME is a motif discovery method to optimize a position weight matrix (PWM). An illustrated drawback being that it needs to exhaustively search the width parameter, and thus could not locate the mutated patterns, particularly those with insertion errors. As also shown in FIG. 7, APCn first discovers patterns from a set of sequences, then clusters the patterns by hierarchical clustering. An illustrated drawback being that it requires the users to input the range of width of patterns. Also, it generally needs users to input the minimum occurrence of patterns, and thus it could not locate patterns with mutations, particularly those with just one or two occurrences. As also shown in FIG. 7, PD-APCn discovers the seed patterns, i.e. patterns with short width, and then extends the seed patterns by jumping over the breakpoint gaps where one or more mutations take place. The APC can then be grown by extending the seed patterns. The final one or more APCs are composed of (aligned) patterns as well as mutation patterns.

In an example experiment conducted by the present inventors, the effectiveness of the present embodiments was demonstrated. Particularly, experiments were conducted to evaluate the performance of the system 100 with respect to how effective it is at discovering and locating conserved functional regions scattered in a dataset with various conserved and mutational patterns synthetically generated. Three sets of synthetic data of different sizes subjecting to different mutations and noise levels were generated randomly. The present embodiments were compared to other approaches quantitatively through a set of metrics, and also applied on two real protein sequence datasets, Cytochrome c and Ubiquitin.

For the purpose of this experiment, three synthetic protein sequence datasets were generated. Dataset 1 was a synthetic dataset composed of 500 protein sequences, generated by: (1) 500 protein sequences were randomly generated at a random length of 50 to 150 under a uniform distribution of the 20 amino acids; (2) a protein segment with 30 amino acids “MKCSQCHTVEKGGKHKTGPNLHGLFGRKTG” extracted from Human Cytochrome C (UniProt KB ID: P99999, positions 12 to 41) was used as the conserved pattern extracted from a real biological dataset; and (3) this pattern was implanted at randomly generated positions among the 500 protein sequences with its position in all sequences recorded. To simulate mutational degeneracy, during the insertion of the conserved pattern, each of its position would undergo 5% chance of substitution, insertion and deletion mutation. Dataset 2 was a synthetic dataset composed of 1000 protein sequences, generated similar to Dataset 1 but double in size. Dataset 3 was a synthetic dataset composed of 2000 protein sequences. The first 1000 sequences were generated the same way as Dataset 1. An additional 1000 protein sequences were randomly generated with variable length of 50 to 150 under an uniform distribution of the 20 amino acids.

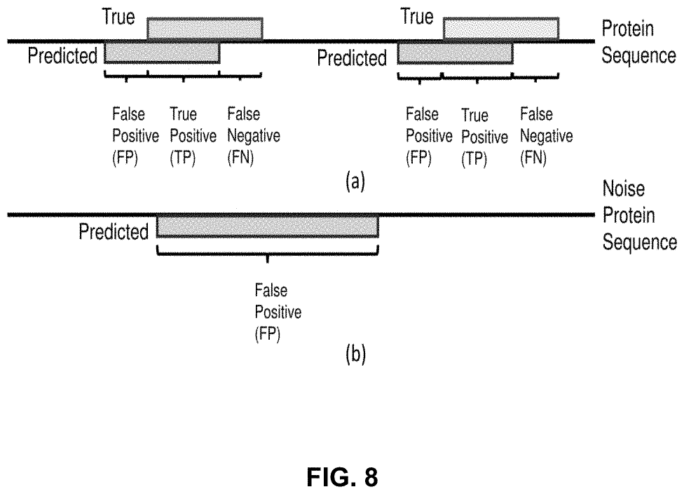

The conserved region positions are a priori known and were considered as the ground-truth. The discovered conserved regions outputted could then be compared with the ground-truth quantitatively. Hence, True Positive (TP), False Positive (FP) and False Negative (FN) could be defined. TP refers to the conserved region positions overlapping with the predicted positions. FP refers to the predicted positions not overlapping with any conserved region positions. Also, any predicted positions on the noise protein sequences are considered as FP. FN refers to the conserved region positions not overlapping with any predicted positions. FIG. 8 provides a graphical illustration of the definition of TP, FP and FN for the quantitative evaluation of the predicted conserved regions. In this experiment on synthetic datasets, the conserved region positions on a protein sequence were priory known. Based on TP, FP and FN, Precision, Recall and Fmeasure could be defined:

$$Precision = \frac{nTP}{nTP + nFP}$$

$$Recall = \frac{nTP}{nTP + nFN}$$

$$Fmeasure = \frac{2 \times Precision \times Recall}{Precision \times Recall}$$

where nTP refers to the total number of TP, nFP refers to the total number of FP, and nFN refers to the total number of FN. Also, if both Precision and Recall are zero, Fmeasure is defined as zero.

In this experiment, both MEME and PD-APCn of the present embodiments were applied to discover the conserved regions from the input protein sequences. In the present example experiment, three options for MEME were used by setting the number of motifs to search to be 1 (nMotifs=1) or 2 (nMotifs=2) or 3 (nMotifs=3). The other MEME parameters remained default.

MEME and PD-APCn were applied on Dataset 1. For MEME, three parameter settings were used, i.e. the number of motifs to search to be 1 (nMotifs=1) or 2 (nMotifs=2) or 3 (nMotifs=3). For PD-APCn, the seed (pattern) width (w_{seed}) was fixed to be 3 and the breakpoint gap (gap_{break}), being the distance of the breakpoint, was varied to be 2 and 3. TABLE 1 summarizes the experimental results on Dataset 1:

TABLE 1

| | Precision | Recall | Fmeasure |
|---|---|---|---|
| MEME [10] (nMotifs = 1) | 0.99839 | 0.49630 | 0.66301 |
| MEME [10] (nMotifs = 2) | 0.99261 | 0.77936 | 0.87315 |
| MEME [10] (nMotifs = 3) | 0.99269 | 0.78816 | 0.87868 |
| PD-APCn (w_{seed} = 3, gap_{break} = 2) | 0.96348 | 0.89905 | 0.93015 |
| PD-APCn (w_{seed} = 3, gap_{break} = 3) | 0.96335 | 0.91655 | 0.93942 |

For Dataset 1, it was observed that MEME obtained a high precision but a low recall. For MEME (nMotifs=1), the precision was 0.99839 but the recall was merely 0.49630, indicating that a significant portion of patterns were not discovered. For MEME (nMotifs=2), the precision increased to 0.99261 and the recall also increased to 0.77936. For MEME (nMotifs=3), the precision further increased to 0.99269 and the recall further increased to 0.78816, but on both cases the marginal increase was lower. For PD-APCn, it obtained a higher level of Fmeasure, outperforming MEME. For PD-APCn (w_{seed}=3, gap_{break}=2), the obtained precision was 0.96348 and the recall was 0.89905. For PD-APCn (w_{seed}=3, gap_{break}=3), the obtained precision slightly decreased to 0.96335 but the recall increased to 0.91655, indicating that a significant portion of patterns were discovered. For both cases, PD-APCn obtained a slightly lower precision but a significantly higher level of recall, thus leading to a higher level of Fmeasure.

MEME and PD-APCn were also applied on Dataset 2. TABLE 2 summarizes the experimental results on Dataset 2:

TABLE 2

| | Precision | Recall | Fmeasure |
|---|---|---|---|
| MEME [10] (nMotifs = 1) | 0.97967 | 0.39232 | 0.56028 |
| MEME [10] (nMotifs = 2) | 0.97922 | 0.84919 | 0.90958 |
| MEME [10] (nMotifs = 3) | 0.97930 | 0.85249 | 0.91151 |
| PD-APCn (w_{seed} = 3, gap_{break} = 2) | 0.96541 | 0.89065 | 0.92092 |
| PD-APCn (w_{seed} = 3, gap_{break} = 3) | 0.96462 | 0.91266 | 0.93792 |

Similar to the results in Dataset 1, PD-APCn obtained a higher level of Fmeasure, outperforming MEME in this dataset. For PD-APCn (w_{seed}=3, gap_{break}=3), it obtained the highest Fmeasure as 0.93792 in this dataset. Again, this high recall indicated that a significant portion of patterns were discovered. These results also demonstrated that scaling up the dataset two times larger did not affect the performance of PD-APCn.

MEME and PD-APCn were also applied on Dataset 3. TABLE 3 summarizes the experimental results on Dataset 2:

TABLE 3

| | Precision | Recall | Fmeasure |
|---|---|---|---|
| MEME [10] (nMotifs = 1) | 0.99898 | 0.48957 | 0.65711 |
| MEME [10] (nMotifs = 2) | 0.99261 | 0.77936 | 0.87315 |
| MEME [10] (nMotifs = 3) | 0.93682 | 0.83278 | 0.88426 |
| PD-APCn (w_{seed} = 3, gap_{break} = 2) | 0.92997 | 0.89605 | 0.91269 |
| PD-APCn (w_{seed} = 3, gap_{break} = 3) | 0.93039 | 0.91266 | 0.92149 |

For Dataset 3, MEME obtained a high precision but a low recall, indicating a large portion of patterns was not discovered. PD-APCn obtained a higher Fmeasure, outperforming MEME, consistently indicating that a significant portion of patterns were discovered. This consistent high recall indicated that PD-APCn has discovered a greater significant portion of patterns than MEME. The top three PWMs outputted by MEME had a width of 15, 8 and 11 respectively; with the third one having substantial overlap with the first two. The top APC outputted by PD-APCn had a width of 35. The top APC captured the entire protein segment introduced in Dataset 3, i.e. “MKCSQCHTVEKGGKHKTGPNLHGLFGRKTG” with 30 amino acids. It is clear from this example experiment that the present embodiments are superior in reflecting aligned protein segment; which explains the superiority in its recalls.

In addition to performance in pattern discovery, runtime was also improved. In this example experiment conducted on a laptop computer (i7-4700HQ CPU 2.4 GHz, 16.0 GB RAM), for Dataset 1 (500 protein sequences), MEME took at least 300 s while PD-APCn took at most 6 s. MEME (nMotifs=3) took 570.683 s to complete running to obtain its optimal Fmeasure (0.87868), while PD-APCn (seed width=3, breakpoint gap=3) took a much less time, 4.843 s, but obtained an even higher Fmeasure (0.93942). It was a speed up of 117.84×. In Dataset 2 (1000 protein sequences), MEME took at least 2000 s while PD-APCn took at most 15 s. MEME (nMotifs=3) took 3155.81 s to complete running to obtain its optimal Fmeasure (0.91151), while PD-APCn (seed width=3, breakpoint gap=3) took a much less time, 12.299 s, but obtained an even higher Fmeasure (0.93792). It was a speed up of 256.59×. In Dataset 3 (2000 protein sequences), MEME took at least 15000 s while PD-APCn took at most 34 s. MEME (nMotifs=3) took 18786.427 s to complete running to obtain its optimal Fmeasure (0.88426), while PD-APCn (seed width=3, breakpoint gap=3) took a much less time, 28.232 s, but obtained an even higher Fmeasure (0.92149); which was a speed up of 665.43×.

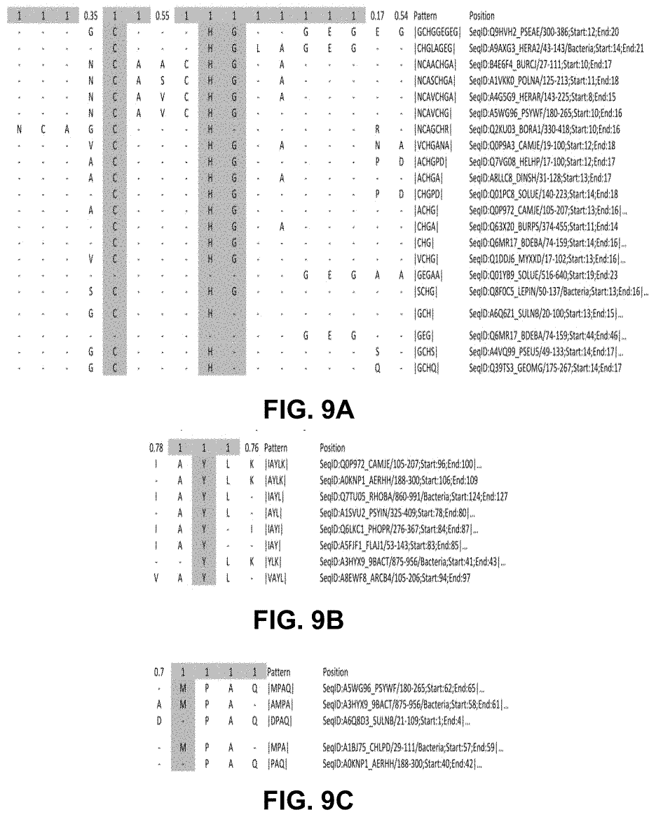

PD-APCn was also performed on a real dataset Cytochrome C, which is a heme-containing protein. It is an essential component of the electron transport chain in the mitochondria, where the heme group plays an important role in accepting and transferring electrons. Applying PD-APCn on the Dataset Cytochrome C, the first three APCs obtained are shown in FIGS. 9A, 9B, and 9C respectively. The 1st APC has covered Cys (C) 14, Cys (C) 17 and His (H) 18. His (H) 18 forms an axial ligand with the heme from the proximal front, i.e. the proximal heme binding site. Cys (C) 14 and Cys (C) 17 enhance and maintain the axial ligand between His18 and the heme. The 2nd APC has covered Tyr (Y) 97, which provides a hydrophobic environment for the function of Cytochrome C. The 3rd APC has covered Met (M) 80 which forms an axial ligand with the heme from the distal side, i.e. the distal heme binding site. These results validate the capability of PD-APCn to discover functional regions in real protein sequences when compared to the Pfam Hidden Markov Model (HMM) of Cytochrome C.

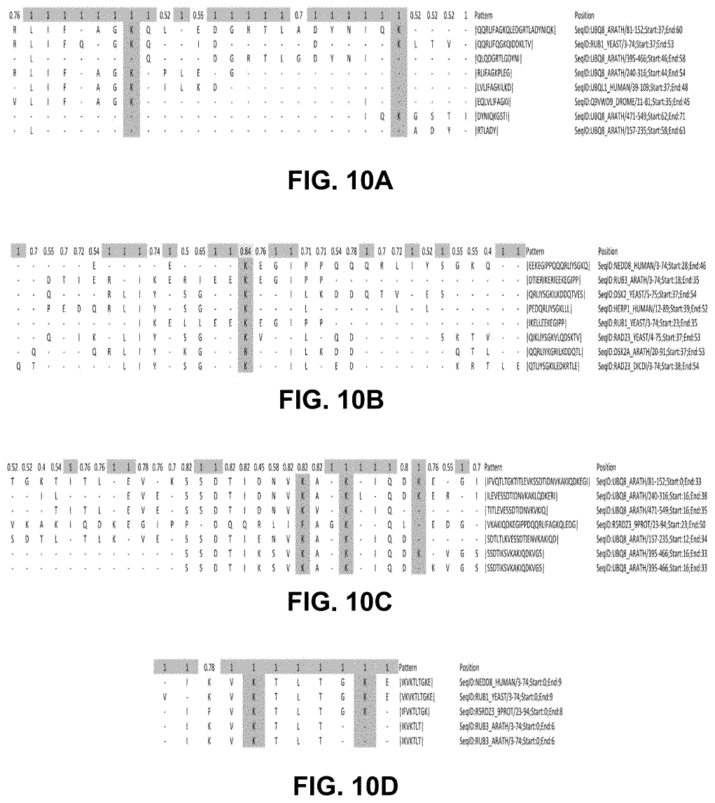

PD-APCn was also performed on a real dataset Ubiquitin, which plays an important role in a process called ubiquitination, where ubuiquitin is attached to a substrate protein. Ubiquitin could either be a single ubiquitin protein or a chain of ubiquitin. To form a chain, an ubiquitin connects to another ubiquitin by binding its C-terminal tail to one of the seven lysine (K) amino acid of its linking partner. The seven lysine (K) are Lys (K) 6, Lys (K) 11, Lys (K) 27, Lys (K) 29, Lys (K) 33, Lys (K) 48 and Lys (K) 63. Applying PD-APCn on the Dataset Ubiquitin C, the first four APCs obtained are shown in FIGS. 10A, 10B, 100, and 10D respectively. The 1st APC has covered Lys (K) 48 and Lys (K) 63. The 2nd APC has covered Lys (K) 33. The 3rd APC has covered Lys (K) 27, Lys (K) 29 and Lys (K) 33. The 4th APC has covered Lys (K) 6 and Lys (K) 11. Hence, all seven lysine (K) have been covered, where they are important for the formation of ubiquitin chains. These results have further validated the capability of PD-APCn to discover functional regions in real protein sequences.

Although the invention has been described with reference to certain specific embodiments, various modifications thereof will be apparent to those skilled in the art without departing from the spirit and scope of the invention as outlined in the claims appended hereto.

1. A computer-implemented method for automated sequence determination using pattern-directed aligned pattern clustering, comprising:
receiving as input one or more character sequences;
determining a set of seed patterns having a predetermined width from each of the character sequences;
generating an address table mapping the determined sets of seed patterns to occurrences of sequences of seed patterns in each of the character sequences;
determining a breakpoint gap between two respective seed patterns in an occurrence of one of the seed pattern sequences in the address table, where a breakpoint gap is present if the gap between the two seed patterns is greater than or equal to a defined non-negative integer;
for each sequence of seed patterns in the address table, where there is a breakpoint gap, merging the respective seed patterns in the sequence surrounding the breakpoint gap with the breakpoint gap into an extended seed pattern, otherwise merging the respective seed patterns in the sequence into an extended seed pattern; and
outputting each of the extended seed patterns.

2. The method of claim 1, further comprising:
determining mutant patterns from the extended seed patterns by comparing extended seed patterns having at least one breakpoint gap to the extended seed patterns without at least one breakpoint gap; and
outputting the mutant patterns.

3. The method of claim 1, wherein the predetermined width is between two and four.

4. The method of claim 1, wherein determining the set of seed patterns having the predetermined width comprises using a pattern discovery approach based on a suffix tree.

5. The method of claim 1, wherein the address table comprises sequences of seed patterns only where the occurrences of those seed patterns are greater than or equal to a predetermined support threshold.

6. The method of claim 5, wherein the predetermined support threshold is determined by determining support of the seed patterns having the predetermined width, sorting such seed patterns in descending order, and setting the predetermined support threshold to be the support of the ninetieth-percentile of the sorted seed patterns.

7. The method of claim 1, wherein mutant patterns are patterns with occurrences less than the predetermined support threshold.

8. The method of claim 1, further comprising outputting a type of each of the mutant patterns by:
where one or more of the characters in the extended seed pattern having at least one breakpoint gap are a different letter compared to the extended seed patterns without at least one breakpoint gap, outputting a substitution mutation;
where one or more of the characters in the extended seed pattern having at least one breakpoint gap are missing compared to the extended seed patterns without at least one breakpoint gap, outputting a deletion mutation; and
where one or more of the characters in the extended seed pattern having at least one breakpoint gap are added compared to the extended seed patterns without at least one breakpoint gap, outputting an insertion mutation.

9. The method of claim 8, further comprising ranking the extended seed patterns according to statistical significance.

10. The method of claim 9, further comprising outputting a set of growing Aligned Pattern Clusters (gAPCs) by:
determining a seed gAPC as the extended seed pattern having the highest-ranking, the seed gAPC comprising the seed patterns and the mutant patterns from the extended seed pattern;
inducing a data space of the seed gAPC using the seed patterns and the mutant patterns;
repeatedly growing the seed patterns and the mutant patterns in the seed gAPC until a termination condition has been reached, by:
if a next highest-ranking extended seed pattern is significantly similar to one or more respective gAPCs in the set of gAPCs and the occurrence of such extended seed pattern is greater than or equal to the predetermined support threshold, the extended seed pattern is included in the respective gAPC that is most similar;
otherwise if the next highest-ranking extended seed pattern is significantly similar to a respective one of the gAPCs in the set of gAPCs and the occurrence of such extended seed pattern is less than the predetermined support threshold, the extended seed pattern is included in the respective gAPC that is most similar to; and
otherwise the next highest-ranking extended seed pattern is included in a new seed gAPC in the set of gAPCs where the new seed gAPC comprises the extended seed patterns and the mutant patterns from the next highest-ranking extended seed pattern; and
outputting the set of gAPCs.

11. A system for automated sequence determination using pattern-directed aligned pattern clustering, the system comprising one or more processors and one or more storage devices storing instructions that, when executed by the one or more processors, cause the one or more processors to execute:
an input module to receive as input one or more character sequences;
a pattern module to determine a set of seed patterns having a predetermined width, to generate an address table mapping the determined sets of seed patterns to occurrences of sequences of seed patterns in each of the character sequences where the occurrences are greater than or equal to a predetermined support threshold, and to determine a breakpoint gap between two respective seed patterns in an occurrence of one of the sequences in the address table, where a breakpoint gap is present if the gap between the two seed patterns is greater than or equal to a defined non-negative integer;
an extension module to, for each sequence of seed patterns in the address table, where there is a breakpoint gap, merge the respective seed patterns in the sequence surrounding the breakpoint gap with the breakpoint gap into an extended seed pattern, otherwise merging the respective seed patterns in the sequence into an extended seed pattern; and
an output module to output each of the extended seed patterns.

12. The system of claim 11, the extension module further determines mutant patterns from the extended seed patterns by comparing extended seed patterns having at least one breakpoint gap to the extended seed patterns without at least one breakpoint gap, and the output module further outputs the mutant patterns.

13. The system of claim 11, wherein the predetermined width is between two and four.

14. The system of claim 11, wherein determining the set of seed patterns having the predetermined width comprises using a pattern discovery approach based on a suffix tree.

15. The system of claim 11, wherein the address table comprises sequences of seed patterns only where the occurrences of those seed patterns are greater than or equal to a predetermined support threshold.

16. The system of claim 15, wherein the predetermined support threshold is determined by determining support of the seed patterns having the predetermined width, sorting such seed patterns in descending order, and setting the predetermined support threshold to be the support of the ninetieth-percentile of the sorted seed patterns.

17. The system of claim 11, wherein mutant patterns are patterns with occurrences less than the predetermined support threshold.

18. The system of claim 11, wherein the extension module further outputs a type of each of the mutant patterns by:
where one or more of the characters in the extended seed pattern having at least one breakpoint gap are a different letter compared to the extended seed patterns without at least one breakpoint gap, outputting a substitution mutation;
where one or more of the characters in the extended seed pattern having at least one breakpoint gap are missing compared to the extended seed patterns without at least one breakpoint gap, outputting a deletion mutation; and
where one or more of the characters in the extended seed pattern having at least one breakpoint gap are added compared to the extended seed patterns without at least one breakpoint gap, outputting an insertion mutation.

19. The system of claim 18, wherein the extension module further ranks the extended seed patterns according to statistical significance.

20. The system of claim 19, the one or more processors further execute a gAPC module to output a set of growing Aligned Pattern Cluster (gAPCs) by:
determining a seed gAPC as the extended seed pattern having the highest-ranking, the seed gAPC comprising the seed patterns and the mutant patterns from the extended seed pattern;
inducing a data space of the seed gAPC using the seed patterns and the mutant patterns; and
repeatedly growing the seed patterns and the mutant patterns in the seed gAPC until a termination condition has been reached, by:
if a next highest-ranking extended seed pattern is significantly similar to one or more respective gAPCs in the set of gAPCs and the occurrence of such extended seed pattern is greater than or equal to the predetermined support threshold, the extended seed pattern is included in the respective gAPC that is most similar;
otherwise if the next highest-ranking extended seed pattern is significantly similar to a respective one of the gAPCs in the set of gAPCs and the occurrence of such extended seed pattern is less than the predetermined support threshold, the extended seed pattern is included in the respective gAPC that is most similar to; and
otherwise the next highest-ranking extended seed pattern is included in a new seed gAPC in the set of gAPCs where the new seed gAPC comprises the extended seed patterns and the mutant patterns from the next highest-ranking extended seed pattern,
wherein the output module further outputs the gAPC.
